(19) United States
(12) Patent Application Publication
(10) Pub. No.: US 2013/0178943 A1
(43) Pub. Date: Jul. 11, 2013

(54) SCREW-CAGE FOR FIXING A CUP OF A SHOULDER PROSTHESIS

(71) Applicant: Marc Duport, Toulouse (FR)

(72) Inventor: Marc Duport, Toulouse (FR)

(21) Appl. No.: 13/823,590

(22) PCT Filed: Sep. 15, 2011
(86) PCT No.: PCT/FR2011/052115

§ 371 Date: Mar. 14, 2013

(30) Foreign Application Priority Data
Sep. 17, 2010 (FR) 10/57437

(51) Int. Cl. A61F 2/40

(52) U.S. Cl. CPC A61F 2/4014
USPC 623/19.14

(57) ABSTRACT

The invention relates to a device (2) for fixing a cup (3) in a bone (21), said device comprising a tubular body (17) extending along a longitudinal axis (A) between a proximal end (18) for co-operating with the cup (3) or a coupling body (9), and a distal end (19) to be fixed in the bone (21), the tubular body (17) defining an inner peripheral surface (24) and an outer peripheral surface (25) provided with screw means. The screw means extend from the proximal end (18) to an intermediate region (27) of the outer peripheral surface (25) arranged at a distance from the proximal (18) and distal (19) ends, and the inner peripheral surface (24) defines a female assembly portion (28) extending from the proximal end (18) in order to co-operate with a male assembly portion of the cup (3) or the coupling body (9).

### TECHNICAL FIELD

The present invention relates to a device for fixing a prosthesis.

### RELATED ART

In France, approximately 10,000 shoulder prostheses are placed each year, including total shoulder prostheses, called standard prostheses, reverse prostheses, and more recently resurfacing prostheses.

This figure, which shows a clear increase in recent years, is in particular due to the increase in the number of pathologies for which the placement of a shoulder prosthesis is now indicated.

In this context, prosthetic shoulder surgery remains a very specialized, highly technical surgery, requiring a long learning period and the use of ancillaries that are often complex. It is therefore desirable to simplify these ancillaries so as to facilitate the procedure, make it more reproducible, and therefore decrease postoperative complications. Such a simplification of the ancillary and the procedure may also have the advantage of decreasing the costs of those operations to place shoulder prostheses.

Several types of shoulder prosthesis exist. Certain traditional prostheses, like that described in US 2004/153161, are fixed on the humerus, using a humeral rod, after a resulting resection of the bone. The installation of a humeral rod requires a significant recess in the bone to receive it, thereby decreasing the bone capital and making the bone more fragile. The placement of such a prosthesis is long and complex. After the rod is installed in the recess, the space between the bone and the humeral rod is generally filled with cement. With time, the cement may become dislocated and no longer ensure correct fixing of the rod.

Resurfacing prostheses also exist, like that described in FR 2,928,827, which are placed by epiphysis of the humerus after potential light planing of the surface of the epiphysis. Such prostheses are not necessarily anchored in the bone as previously described.

When a simple resurfacing of the epiphysis is not indicated, for example if the bone is too fragile, the surgeon may choose to use an intermediate so-called “half-resurfacing” technique where part of the epiphysis is resected. The surgeon is then called upon to use a prosthesis like that described in document EP 0,538,895, including a cup designed to replace the part of the epiphysis that is resected and an anchoring screw designed to be fixed in the bone by screwing, such an anchoring screw including a threading over its entire length for that purpose. The cup is then fixed on the anchoring screw.

The positioning of the anchoring screw conditions the positioning of the cup and must be as precise as possible. The patient may experience discomfort or pain because the prosthesis was poorly positioned, which then requires the surgeon to re-operate to reposition the prosthesis correctly. The removal of the anchoring screw, which is designed to provide solid anchoring of the prosthesis in the bone, can cause problems and make the bone more fragile.

Document GB 2,199,626 describes a dental implant with a generally tubular shape having an openwork lower area, a threaded central portion, and a smooth upper portion forming a head.

Document US 2010/191340 describes prosthetic head for a humerus including a cup designed to be equipped with a tubular screw including a threading extending over its entire length.

### BRIEF SUMMARY

The present invention aims to resolve all or part of the various aforementioned Drawbacks.

In that context, the present invention aims to propose a device for fixing a cup that preserves the bone capital of the patient and ensures improved, precise and quick fixing of the cup, while being able to be removed without making the bone more fragile.

To that end, the present invention relates to a device for fixing a cup, the device being designed to be implanted in a bone, in particular a humeral epiphysis, the device comprising a tubular body extending along a longitudinal axis between a proximal end for cooperating with the cup or a coupling body, and an opposite distal end to be fixed in the bone, the tubular body defining an inner peripheral surface and an outer peripheral surface provided with screw means to allow the device to be screwed into the bone, the device being characterized in that the screw means include a threading extending substantially from the proximal end to an intermediate region of the outer peripheral surface arranged at a distance from the proximal and distal ends, in that the threading has a variable screw pitch increasing from the proximal end toward the intermediate region, and in that the inner peripheral surface defines a female assembly portion extending from the proximal end in order to cooperate with a male assembly portion of the cup or with a male intermediate assembly portion of the coupling body.

Thus, a device according to the invention makes it possible to fix a cup, standard or inverted, in the event of bone fragility and when it is not possible to perform a simple resurfacing. When the tubular body of the device is fixed in the bone, it ensures correct anchoring of the device owing to the screw means and the cup is assembled directly or indirectly on the device owing to the cooperation of the complementary assembly portions. The placement procedure is simplified, in particular since the coupling of the cup on the device is simple. The fixing of the device is done simply as follows: an orifice with a reduced length, corresponding to the length between the distal end and the intermediate region, is formed in the bone, then the part of the outer peripheral surface provided without screw means (i.e., the part of the outer peripheral surface situated between the distal end and the intermediate region) is inserted into the orifice, and the complete insertion of the device in the bone is then obtained owing to the screw means. During screwing, part of the bone is inserted into the tubular body and therefore makes it possible to preserve bone capital. The screw means ensure suitable fixing of the device in the bone, while the part of the outer peripheral surface provided without screw means participates in aligning and maintaining the device in the bone. Furthermore, during screwing of the device, the variation of the screw pitch creates compacting of the bone and improves fixing of the device.

If needed, the device may be removed using a hole saw whereof the inner diameter is suitable for encompassing the screw means. With the hole saw, the part of the bone cooperating with the screw means is cut. Given that the screw means do not extend over the entire length of the device, but only over part thereof, the length of the cut to be made with the hole saw is reduced relative to a traditional anchoring screw. Thus, the bone capital is preserved and the risks of making the bone more fragile are reduced in case of removal. The screw means therefore facilitate removal of the device in the event of a change in prosthesis.

According to one embodiment, the female assembly portion has a female tapered shape. A tapered shape is easy to produce and makes it possible to perform a quick assembly while allowing a correct alignment of the cup with the anchoring device.

According to one possibility, the height of the thread of the threading increases from the proximal end toward the intermediate region. The cooperation surface between the bone and the threading increases with the height of the thread and thereby improves the fixing of the device in the bone.

According to one embodiment, the intermediate region is situated at a distance from the proximal end equal to a distance comprised between 20 and 70% of the distance between the proximal and distal ends, preferably comprised between 40 and 50%.

According to one embodiment, the tubular body has a distal surface connecting the inner and outer peripheral surfaces and forming a bevel. The bevel makes it possible to facilitate the penetration of the device in the bone during its screwing.

According to one possibility, the part of the outer peripheral surface extending from the intermediate region to the distal end is substantially smooth. Thus, such a smooth surface makes it possible to facilitate the removal of the device without making the bone more fragile.

According to one feature of the invention, the part of the outer peripheral surface extending from the intermediate region to the distal end has a least one orifice passing through the tubular body. Such an orifice allows better re-habitation of the bone when it grows back to secure the part of the bone in contact with the inner peripheral surface to the part of the bone in contact with the outer peripheral surface, in particular without compromising any removal.

The invention also relates to a prosthesis comprising a device according to the invention and a cup including a male assembly portion designed to cooperate directly or indirectly with a female assembly portion of the device.

According to one embodiment, the prosthesis also includes a coupling body designed to be arranged between the device according to the invention and the cup, the coupling body including a male intermediate assembly portion designed to cooperate by shape matching with the female assembly portion of the device and a female intermediate assembly portion designed to cooperate by shape matching with the male assembly portion of the cup.

### BRIEF DESCRIPTION OF THE DRAWINGS

The invention will be well understood using the following detailed description thereof provided in light of the appended drawing, showing, as one non-limiting example, one embodiment of the device, and in which:

FIG. 1 is a diagrammatic perspective view of the various elements of the prosthesis;

FIG. 2 is a diagrammatic partial cross-sectional and perspective view of the prosthesis of FIG. 1;

FIG. 3 is a diagrammatic perspective view of a prosthesis of FIG. 1 implanted in a humeral epiphysis.

### DETAILED DESCRIPTION

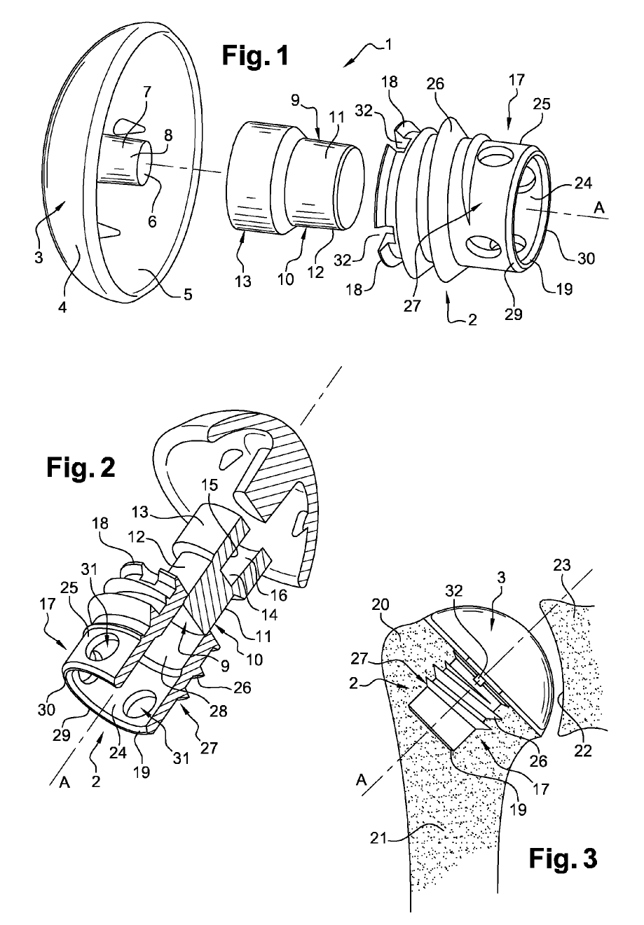

A prosthesis 1, illustrated in FIG. 1, includes a device 2 and a cup 3 designed to be assembled in the device 2.

The cup 3 has a hollow spherical cap 4, having an inner face 5 and a tapered central stem 6 protruding from the center of the inner face 5 of the spherical cap 4. The central stem 6 delimits a male assembly portion 7 forming a male Morse cone 8.

Optionally, as is the case in the embodiment illustrated in figures, the prosthesis 1 also comprises a coupling body 9. This coupling body 9 includes a first portion 10 with a substantially tapered shape having a male intermediate assembly portion 11 forming a male Morse cone 12. The coupling body 9 also includes a second tubular portion 13 provided with a cavity 14, illustrated in FIG. 2, that is substantially tapered and defines a female intermediate assembly portion 15. The female intermediate assembly portion 15 forms a female Morse cone 16 sized to cooperate by shape matching with the male assembly portion 7 of the cup 3. According to the embodiment illustrated in the figures, the coupling body 9 has a male Morse cone 12 and a female Morse cone 16 whereof the respective axes of symmetry are combined. Alternatively and not shown, the male Morse cone 12 and the female Morse cone 16 can have separate respective axes of symmetry that are offset and/or not parallel.

The device 2 includes a tubular body 17 extending along a longitudinal axis A between a proximal end 18, designed to cooperate directly or indirectly with the cup 3 or the coupling body 9, and an opposite distal end 19, designed to be fixed in an epiphysis 20 of the bone 21, illustrated in FIG. 3. This bone 21 corresponds to a humerus when the cup 3 is designed to be arranged in a glenoid cavity 22 of a scapula 23 partially illustrated in FIG. 3.

The tubular body 17 defines an inner peripheral surface 24, illustrated in FIG. 2, and an outer peripheral surface 25 provided with screw means, made in the form of a threading 26, to allow the device 2 to be screwed in the bone 21.

The threading 26 extends substantially from the proximal end 18 to an intermediate region 27 of the outer peripheral surface 25 situated at a distance from the proximal 18 and distal 19 ends. This intermediate region 27 is situated at a distance from the proximal end 18 equal to a distance comprised between 20 and 70% of the distance between the proximal 18 and distal 19 ends.

The threading 26 has a variable pitch that increases along the threading 26 from the proximal end 18 toward the intermediate region 27. The height of the thread of the threading 26 also increases following the threading 26 from the proximal end 18 toward the intermediate region 27.

The inner peripheral surface 24 defines a female assembly portion 28 that extends substantially from the proximal end 18 and that is sized to cooperate by shape matching with the male intermediate assembly portion 11 of the coupling body 9. In one embodiment not shown in the figures, the female assembly portion 28 is sized to cooperate by shape matching with the male assembly portion 7 of the cup 3.

The tubular body 17 has a distal surface 29 connecting the inner 24 and outer 25 peripheral surfaces. This distal surface 29 forms a bevel 30 that makes it possible to facilitate the insertion of the device 2 into the bone 21 by screwing.

A part of the outer peripheral surface 25 that extends from the intermediate region 27 to the distal end 19 is substantially smooth and has four orifices 31 passing through the tubular body 17.

Lastly, the device 2 includes four notches 32 formed in the threading 26, at the proximal end 18. These notches 32 are designed to cooperate with a screwing tool and facilitate the operation screwing the device 2 in the bone 21.

Thus, a device 2 according to the invention described above makes it possible to fix the cup 3. When the tubular body 17 of the device 2 is fixed in the bone, the correct anchoring of the device 2 is ensured owing to the threading 26. The placement procedure is simplified, since the coupling of the cup 3 on the device 2 is simple.

If needed, the device 2 may be removed using a hole saw in the manner previously described. Owing to the device 2, the part of the bone 21 cooperating with the threading 26 and cut may be reduced, since the threading 26 does not extend over the entire length of the device 2. In the case where the device 2 is removed, the bone capital is preserved and the risks of making the bone 21 more fragile are reduced.

Of course, the example embodiment mentioned above is not limiting, and other details and improvements may be made to the device 2 according to the invention, without going beyond the scope of the invention, where other forms of the device 2 may be considered.

1. A device for fixing a cup, the device being designed to be implanted in a bone, in particular a humeral epiphysis, the device comprising a tubular body extending along a longitudinal axis between a proximal end for cooperating with the cup or a coupling body, and an opposite distal end to be fixed in the bone, the tubular body defining an inner peripheral surface and an outer peripheral surface provided with screw means to allow the device to be screwed into the bone, wherein the screw means include a threading extending substantially from the proximal end to an intermediate region of the outer peripheral surface arranged at a distance from the proximal and distal ends, wherein the threading has a variable screw pitch increasing from the proximal end toward the intermediate region, and wherein the inner peripheral surface defines a female assembly portion extending from the proximal end in order to cooperate with a male assembly portion of the cup or with a male intermediate assembly portion of the coupling body.

2. The device according to claim 1, wherein the female assembly portion has a female tapered shape.

3. The device according to claim 1, wherein the height of the thread of the threading increases from the proximal end toward the intermediate region.

4. The device according to claim 1, wherein the intermediate region is situated at a distance from the proximal end equal to a distance comprised between 20 and 70% of the distance between the proximal and distal ends.

5. The device according to claim 1, wherein the tubular body has a distal surface connecting the inner and outer peripheral surfaces and forming a bevel.

6. The device according to claim 1, wherein the part of the outer peripheral surface extending from the intermediate region to the distal end is substantially smooth.

7. The device according to claim 1, wherein the part of the outer peripheral surface extending from the intermediate region to the distal end has a least one orifice passing through the tubular body.

8. A prosthesis comprising a device according to claim 1 and a cup including a male assembly portion designed to cooperate directly or indirectly with a female assembly portion of the device.

9. The prosthesis according to claim 8, also including a coupling body designed to be arranged between the device and the cup, the coupling body including a male intermediate assembly portion designed to cooperate by shape matching with the female assembly portion of the device and a female intermediate assembly portion designed to cooperate by shape matching with the male assembly portion of the cup.
